(19) United States
(12) Patent Application Publication
(10) Pub. No.: US 2023/0225388 A1
(43) Pub. Date: Jul. 20, 2023

(54) PROTEIN COMPOSITIONS PRODUCED FROM HEMP PLANT MATERIALS

(71) Applicant: BOTANECO INC., Calgary (CA)

(72) Inventors: Cesar BIRABONEYE, Calgary (CA); Shuanghui LIU, Calgary (CA); Jason HARGREAVES, Calgary (CA); Abhimanyu JAYAKUMAR, Calgary (CA); Saskia TERSTEEG, Calgary (CA)

(73) Assignee: BOTANECO INC., Calgary, AB (CA)

(21) Appl. No.: 18/011,520

(22) PCT Filed: Jun. 18, 2021
(86) PCT No.: PCT/CA2021/050840

§ 371 Date: Dec. 19, 2022

Related U.S. Application Data
(60) Provisional application No. 63/041,370, filed on Jun. 19, 2020.

(51) Int. Cl. A23L 33/185; C07K 14/415; A23L 33/115; A23K 20/147; A23K 20/158; A23K 10/30; A23J 1/14; B01D 61/14; B01D 61/16

(52) U.S. Cl. CPC A23L 33/185; C07K 14/415; A23L 33/115; A23K 20/147; A23K 20/158; A23K 10/30; A23J 1/14; B01D 61/147; B01D 61/145; B01D 61/149; B01D 61/16; B01D 2311/04; B01D 2311/06

(57) ABSTRACT

Disclosed are plant protein compositions prepared from hemp plant material. Hemp seeds are deconstructed and separated into heavy protein-rich fractions and light oil-rich fractions. The deconstruction processes avoid the use of organic solvents and temperatures above 60° C. The separated fractions are then selectively processed to produce selected plant protein containing compositions comprising from about 30% (w/w) to about 95% hemp protein, from about 5% (w/w) and to about 60% (w/w) plant oil. The plant protein compositions from hemp plants are suitable for use as ingredients in nutritional formulations.

### FIELD OF THE DISCLOSURE

The present disclosure generally relates to protein compositions. More specifically, this disclosure pertains to protein compositions recoverable from plant components.

### BACKGROUND

Plant materials are known to be excellent sources of edible oils and proteins. For example, edible oils are commonly produced by pressing under high pressures, seeds harvested from agricultural crops such as Brassica spp., corn, soybeans, peanuts, sunflower, safflower, hemp, flax, legumes, and cotton, among others.

Plant materials with a high oil content (e.g. 35% or more) are typically processed with techniques that have evolved to optimize plant oil yield. In general, these processing techniques involve combinations of mechanical pressing and solvent extraction of selected plant material. After the oil is extracted, the remaining oilseed cake or meal may be used to extract plant proteins as a co-product. Protein extraction processes commonly involve heat treatment to remove extraction solvents from the oilseed cake or meal. However, due to the harsh processing conditions employed, the plant proteins recovered from oilseed cake or meal generally lose their native conformational state and form denatured or fractured structures. Preparations consisting primarily of denatured proteins are less suitable as a nutritional ingredient, since properties such as solubility, flavor, smell and color are generally all negatively impacted by the processing conditions.

Thus, it is clear that while plant materials represent a valuable source of proteins, the quality of protein preparations obtained from these plant materials known to the art is suboptimal.

### SUMMARY

The embodiments of the present disclosure generally relate to protein preparations produced from selected hemp plant materials. According to one aspect, the selected hemp plant materials may be hemp seeds.

One embodiment disclosed herein relates to hemp protein compositions comprising from about 30% (w/w) and to about 95% (w/w) hemp protein (N×6.25), from about 5% (w/w) and to about 60% (w/w) plant oil.

One embodiment disclosed herein relates to hemp protein compositions comprising from about 30% (w/w) and to about 95% (w/w) hemp protein (N×6.25), from about 5% (w/w) and to about 60% (w/w) plant oil, and from up to about 4% (w/w) carbohydrates.

One embodiment disclosed herein relates to hemp protein compositions comprising from about 30% (w/w) and to about 95% (w/w) hemp protein (N×6.25), from about 5% (w/w) and to about 60% (w/w) plant oil, and from about 1% (w/w) and to about 4% (w/w) carbohydrates.

According to another aspect, the hemp protein compositions may comprise from about 34% (w/w) and to about 93% (w/w) hemp protein (N×6.25), from about 7% (w/w) and to about 59% (w/w) plant oil.

According to another aspect, the hemp protein compositions may comprise from about 34% (w/w) and to about 93% (w/w) hemp protein (N×6.25), from about 7% (w/w) and to about 59% (w/w) plant oil, and up to about 4% (w/w) carbohydrates.

According to another aspect, the hemp protein compositions may comprise from about 34% (w/w) and to about 93% (w/w) hemp protein (N×6.25), from about 7% (w/w) and to about 59% (w/w) plant oil, and from about 1% (w/w) and to about 4% (w/w) carbohydrates.

According to another aspect, the hemp protein compositions may be a hemp protein concentrate comprising from at least about 84% (w/w) and less than about 90% (w/w) hemp protein (N×6.25), and from about 11% (w/w) to about 19% (w/w) plant oil.

According to another aspect, the hemp protein concentrate may comprise at least about 85% (w/w) and less than about 90% (w/w) hemp proteins (N×6.25).

According to another aspect, the hemp protein concentrate may comprise at least about 86% (w/w) and less than about 90% (w/w) hemp proteins (N×6.25).

According to another aspect, the hemp protein composition may be a hemp protein isolate comprising at least about 90% (w/w) hemp protein (N×6.25), and from about 6% (w/w) to about 8% (w/w) plant oil.

According to another aspect, the hemp protein isolate may comprise at least about 91% (w/w) hemp proteins (N×6.25).

According to another aspect, the hemp protein isolate may comprise at least about 92% (w/w) hemp proteins (N×6.25).

According to another aspect, the hemp protein isolate may comprise at least about 93% (w/w) hemp proteins (N×6.25).

According to another aspect, the hemp protein composition may be a hemp protein-lipid complex containing mixture comprising from about 34% (w/w) to about 49% (w/w) protein (N×6.25), from about 34% (w/w) to about 59% (w/w) plant oil, and from about 1% (w/w) to about 4% (w/w) carbohydrates.

According to another aspect, the total protein (N×6.25) and plant oil content may range from about 83% to about 100%, and wherein the ratio of protein to oil may range from about 1 to about 10 on a weight-by-weight basis.

According to another aspect, the hemp protein composition may be a hemp protein concentrate comprising a total protein (N×6.25) and plant oil content of about 100% (w/w).

According to another aspect, the hemp protein composition may be a hemp protein isolate comprising a total protein (N×6.25) and plant oil content of about 100% (w/w).

According to another aspect, the hemp protein composition may be a hemp protein-lipid complex containing mixture comprising a total protein (N×6.25) and plant oil content of at least about 83% (w/w).

According to another aspect, the hemp protein composition may be a hemp protein concentrate, wherein the ratio of protein (N×6.25) to oil is at least about 5.3:1 on a weight-by-weight basis.

According to another aspect, the hemp protein composition may be a hemp protein isolate, wherein the ratio of protein (N×6.25) to oil is at least about 14:1 on a weight-by-weight basis.

According to another aspect, the hemp protein composition may be a hemp protein-lipid complex containing mixture, wherein the ratio of protein (N×6.25) to oil is at least about 0.6:1 on a weight-by-weight basis.

According to another aspect, the weight percentage of essential amino acids in the hemp protein compositions may be about 30% (w/w) or more by weight protein.

According to yet another aspect, the lysine content may range from about 2.5% up to about 5% by weight of crude protein of the hemp protein compositions.

Another embodiment disclosed herein relates to methods of making hemp protein compositions, comprising the steps of:
(i) providing seeds from a hemp plant;
(ii) comminuting the seeds in an aqueous solution to obtain a mixture comprising comminuted seed particles having mean particle sizes in a range of about 5 μm to about 200 μm; and
(iii) separating the mixture into a first solid phase and a first liquid phase;

and at least one of:

(A)
(iv) separating the first liquid phase into a light liquid phase and a heavy liquid phase; and
(a)
   (v) treating the heavy liquid phase by microfiltration to recover therefrom a permeate;
   (vi) treating the permeate by ultrafiltration to recover therefrom the protein retentate; and
   (vii) drying the protein retentate to obtain a first protein-lipid complex containing mixture;
   or
(b)
   (viii) precipitating the protein in the heavy liquid phase to obtain a protein precipitate; and
   (ix) drying the protein precipitate to obtain a first protein concentrate;

or

(B)
(x) extracting the first solid phase to obtain a first solid phase extract;
(xi) separating the first solid phase extract to obtain a second solid phase and a second liquid phase; and

(a)
(xii) settling the second liquid phase to obtain a heavy second liquid phase and a light oil-rich phase;
(xiii) precipitating the heavy second liquid phase to recover a protein precipitate; and
(xiv) drying the protein precipitate to obtain a third protein concentrate;
or

(b)
(xv) precipitating the protein in the second liquid phase to recover a protein precipitate; and
(xvi) drying the protein precipitate to obtain a protein isolate;

or

(C)
(xvii) drying the first liquid phase to obtain a second protein-lipid complex containing mixture.

Another embodiment disclosed herein relates to methods of making protein-lipid complex containing mixtures, comprising the steps of:
(i) providing whole dehulled seeds from a hemp plant;
(ii) comminuting the seeds in an aqueous solution to obtain a mixture comprising comminuted seed particles having mean particle sizes in a range of about 5 μm to about 200 μm;
(iii) separating the mixture into a solid phase and a liquid phase;
(iv) separating the liquid phase into a light liquid phase and a heavy liquid phase;
(v) treating the heavy liquid phase by microfiltration to recover therefrom a permeate;
(vi) treating the permeate by ultrafiltration to recover therefrom the protein retentate; and
(vii) drying the protein retentate to obtain a protein-lipid complex containing mixture.

Another embodiment of the present disclosure relates to methods of making hemp protein concentrates, comprising the steps of:
(i) providing whole seeds from a hemp plant;
(ii) comminuting the seeds in an aqueous solution to obtain a mixture comprising comminuted seed particles having mean particle sizes in a range of about 5 μm to about 200 μm;
(iii) separating the mixture into a solid phase and a liquid phase;
(iv) separating the liquid phase into a light liquid phase and a heavy liquid phase;
(v) precipitating the protein in the heavy liquid phase to obtain a protein precipitate; and
(vi) drying the protein precipitate to obtain a protein concentrate.

Another embodiment of the present disclosure relates to methods of making protein-lipid complex containing mixtures, comprising the steps of:
(i) providing whole seeds from a hemp plant;
(ii) comminuting the seeds in an aqueous solution to obtain a mixture comprising comminuted seed particles having mean particle sizes in a range of about 5 μm to about 200 μm;
(iii) separating the mixture into a solid phase and a liquid phase; and
(iv) drying the liquid phase to obtain a protein-lipid complex containing mixture.

According to one aspect, the whole seeds may be soaked for at least 6 hours prior to comminuting the seeds.

Another embodiment of the present disclosure relates to methods of making hemp protein concentrates, comprising the steps of:
(i) providing whole seeds from a hemp plant;
(ii) comminuting the seeds in an aqueous solution to obtain a mixture comprising comminuted seed particles having mean particle sizes in a range of about 5 μm to about 200 μm; and
(iii) separating the mixture into a first solid phase and a first liquid phase;
(iv) precipitating the first solid phase to obtain a second solid phase and a second liquid phase;
(v) extracting the first solid phase to obtain a first solid phase extract;
(vi) separating the first solid phase extract to obtain a second solid phase and a second liquid phase.
(vii) settling the second liquid phase to obtain a heavy second liquid phase and a light oil-rich phase;
(viii) precipitating the heavy second liquid phase to obtain a protein precipitate; and
(ix) drying the protein precipitate to obtain a protein concentrate.

Another embodiment of the present disclosure relates to methods of making hemp protein isolates, comprising the steps of:
(i) providing whole seeds from a hemp plant;
(ii) comminuting the seeds in an aqueous solution to obtain a mixture comprising comminuted seed particles having mean particle sizes in a range of about 5 μm to about 200 μm; and
(iii) separating the mixture into a first solid phase and a first liquid phase;
(iv) extracting the first solid phase to obtain a first solid phase extract;
(vi) separating the first solid phase extract to obtain a second solid phase and a second liquid phase;
(vii) precipitating the protein in the second liquid phase to obtain a protein precipitate; and
(viii) drying the protein precipitate to obtain a protein isolate.

Another embodiment of the present disclosure relates to methods of preparing nutritional formulations, the method comprising:
(i) providing a hemp protein composition produced by the methods disclosed herein, said hemp protein concentrate comprising from about 35% (w/w) to about 96% hemp protein (N×6.25), from about 7% (w/w) and to about 60% (w/w) plant oil;
(ii) providing a formulary ingredient suitable for inclusion in a nutritional formulation; and
(iii) blending together the hemp plant protein composition with the formulary ingredient to form a nutritional formulation comprising the hemp protein composition disclosed herein.

Another embodiment disclosed herein relates to uses of a hemp protein composition as an ingredient for preparing a nutritional formulation, wherein the hemp protein composition comprises from about 30% (w/w) to about 95% hemp protein, from about 5% (w/w) and to about 60% (w/w) plant oil, the balance of the hemp protein composition substantially being constituted by carbohydrates, water, and ash.

Another embodiment disclosed herein relates to nutritional formulations comprising a hemp protein composition comprising at least about 65% (w/w) or more plant protein, from about 30% (w/w) to about 95% hemp protein (N×6.25), from about 5% (w/w) and to about 60% (w/w) plant oil, the balance of the hemp protein composition substantially being constituted by carbohydrates, water, and ash.

Other features and advantages of the present disclosure will become apparent from the following detailed description. It should be understood, however, that the detailed description, while indicating preferred implementations of the present disclosure, is given by way of illustration only, since various changes and modifications within the spirit and scope of the disclosure will become apparent to those of skill in the art from the detailed description.

### BRIEF DESCRIPTION OF THE DRAWINGS

These and other features of the disclosure will become more apparent in the following detailed description in which reference is made to the appended drawings. The figures are provided herein for a better understanding of the example embodiments and to show more clearly how the various example embodiments may be carried into effect. The figures are not intended to limit the present disclosure.

FIG. 1 is a schematic block diagram illustrating an example process 100 according to an example of an embodiment of the present disclosure, for making plant protein compositions.

FIGS. 2A-2B are schematic block diagrams illustrating an example process 200 including an example sub-process 200a (FIG. 2B) according to an example of an embodiment of the present disclosure, for making plant protein compositions.

FIGS. 3A-3C are schematic block diagrams illustrating an example process 300 including example sub-processes 300a (FIG. 3B) and 300b (FIG. 3C) according to an example of an embodiment of the present disclosure, for making plant protein compositions.

FIGS. 4A-4B show scanning electron-microscopic images of the surface morphology of certain protein-lipid complex containing mixtures at a magnification of 120× (FIG. 4A) and 500× (FIG. 4B) with a working distance of 9 mm, respectively. The images are created in accordance to the method of Chang, C., Varankovich, N., Nickerson, M. T., 2016, Microencapsulation of canola oil by lentil protein isolate-based wall materials, Food Chemistry 212:264-273.

The figures together with the following detailed description make apparent to those skilled in this art how the disclosure may be implemented in practice.

### DETAILED DESCRIPTION

As used herein and in the claims, the singular forms, such as “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either”. The term “and/or” is intended to represent an inclusive or. That is “X and/or Y” is intended to mean X or Y or both, for example. As a further example, X, Y and/or Z is intended to mean X or Y or Z or any combination thereof.

When ranges are used herein for physical properties such as molecular weights, chemical properties, chemical formulae, and the like, all combinations and sub-combinations of ranges and specific embodiments therein are intended to be included. Other than in the operating examples or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by the context. Furthermore, any range of values described herein is intended to specifically include the limiting values of the range, and any intermediate value or sub-range within the given range, and all such intermediate values and sub-ranges are individually and specifically disclosed (e.g. a range of 1 to 5 includes 1, 5, and all values therebetween). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only and is not intended to limit the scope of the present disclosure, which is defined solely by the claims.

Unless otherwise defined, scientific and technical terms used in connection with the formulations described herein shall have the meanings that are commonly understood by those of ordinary skill in the art. The terminology used herein is for the purpose of describing particular embodiments only and is not intended to limit the scope of the present disclosure, which is defined solely by the claims.

All publications, patents, and patent applications referred herein are herein incorporated by reference in their entirety to the same extent as if each individual publication, patent or patent application was specifically indicated to be incorporated by reference in its entirety.

The term “hemp” as used herein, refers to a plant belonging to the genus Cannabis, and includes Cannabis sativa, Cannabis indica, and Cannabis ruderalis, and further includes all species, subspecies, cultivars, varieties, hybrids and genotypes.

The term “essential amino acids” as used herein, refers to histidine, isoleucine, leucine, lysine, methionine, phenylalanine, threonine, tryptophan, and valine. It is noted that in at least some fish species including without limitation, salmon, arginine may additionally be considered an essential amino acid.

The term “comminuting” as used herein, refers to a process for deconstructing plant material into particles having sizes in a range from about 5 μm to about 200 μm and sizes therebetween. Examples of comminuting processes include wet milling, grinding, homogenization, and the like. Suitable comminuting equipment includes a seed mill, a colloid mill, a hammer mill, a blade mill, a roller mill, and the like.

The phrase “formulating the protein composition to form a nutritional product” as used herein, refers to mixing or blending a protein composition produced by the methods disclosed herein, at least one other ingredient suitable for inclusion in a nutritional product.

The term “protein concentrate”, as used herein, refers to a composition comprising less than 90% (w/w) protein, and less than 20% (w/w) plant oil.

The term “protein isolate”, as used herein, refers to a composition comprising at least 90% (w/w) protein, and less than 20% (w/w) plant oil.

The term “protein-lipid complex containing mixture”, as used herein, refers to a composition comprising at least 30% plant oil and at least 30% protein.

It is noted that reference may be made herein to various percentages of protein to quantify the amount of protein which may be present in a sample or composition. Such percentages may be expressed as a percentage of the total weight of the sample or composition, for example, 90% (w/w). Those skilled in the art will understand that the protein content in a preparation produced as disclosed herein, can readily be measured by various methodologies known to the art including, for example among others, the Kjeldahl method, or methods for determining measuring nitrogen by combustion disclosed in the Association of Official Analytical Chemists (AOAC) Method 992.23, or methods disclosed in the American Association of Cereal Chemists (AACC) Method 46-30, 1999. Moreover, those skilled in this art will understand that to convert measured nitrogen to protein, a conversion factor may be used. A commonly used conversion factor in this respect is 6.25. The protein content of the compositions set forth in the present disclosure refers to an applied conversion factor of 6.25. Furthermore, those of skilled in this art will understand that due to certain inherent inaccuracies in the foregoing conversion methodology it is possible that when calculating total oil and protein constituents in certain compositions, the calculated total oil and protein (N×6.25) may slightly exceed 100%, and be calculated to be for example 101% or 102%. Thus, for example, when reference is made herein to a sample containing 90% (w/w) protein, such a sample contains 90% (w/w) protein based on the application of a conversion factor of 6.25. The foregoing may be expressed herein as: “a sample containing 90% (w/w) protein (N×6.25)”, or in a substantially similar manner.

In overview, it has surprisingly been realized that protein compositions containing high concentrations of protein moieties, and oil and carbohydrates may be recovered from comminuted whole plant parts of hemp plants, wherein the protein moieties are substantially free of heat or solvent damage and thus, are present in a non-denatured conformational state. The hemp protein compositions produced by the methods disclosed herein may contain substantial quantities of essential amino acids. Furthermore, the hemp compositions disclosed herein exhibit desirable physico-chemical properties including solubility, emulsion stability and oil- and water hydration capacities.

The hemp protein compositions of the present disclosure are useful for the preparation of nutritional formulations including for example, nutritional aquaculture formulations, nutritional animal feed formulations, nutritional poultry formulations, and nutritional formulations suitable for human consumption, among others. Furthermore, the residual presence of plant oils in the present hemp protein compositions may be beneficial as energy sources in nutritional formulations. The presence of plant oils in the present hemp protein compositions may obviate the need for addition of extraneous oil into nutritional feed formulations.

Accordingly, one embodiment of the present disclosure pertains to hemp protein compositions comprising from about 35% (w/w) to about 95% hemp protein (N×6.25), from about 5% (w/w) and to about 60% (w/w) plant oil.

Accordingly, one embodiment of the present disclosure pertains to hemp protein compositions comprising from about 35% (w/w) to about 95% hemp protein (N×6.25), from about 5% (w/w) and to about 60% (w/w) plant oil, and up to about 4% (w/w) carbohydrates.

Accordingly, one embodiment of the present disclosure pertains to hemp protein compositions comprising from about 35% (w/w) to about 95% hemp protein (N×6.25), from about 5% (w/w) and to about 60% (w/w) plant oil, and from about 1% (w/w) and to about 4% (w/w) carbohydrates.

The hemp protein compositions disclosed herein may be prepared from parts of hemp plants, notably seeds, i.e. plants belonging to the plant species Cannabis sativa, Cannabis indica or Cannabis ruderalis, wherein the seeds are produced and harvested by agricultural practices or by horticultural practices. Also suitable are hemp subspecies, varieties, cultivars, genotypes, or hybrids.

Accordingly, another embodiment according to the present disclosure pertains to methods and processes for producing the disclosed hemp plant protein compositions, wherein the methods and processes generally comprise the steps of deconstructing whole seeds of selected plants with a selected comminuting process to produce comminuted mixtures comprising heavy high-protein fractions and light high-oil fractions, separating the heavy high-protein fractions from the light high-oil fractions, and then separately further processing the heavy high-protein fractions and/or the light high-oil fractions to produce the plant protein compositions. The methods disclosed herein avoid the use of organic solvents and high temperatures i.e. temperatures greater than 60° C.

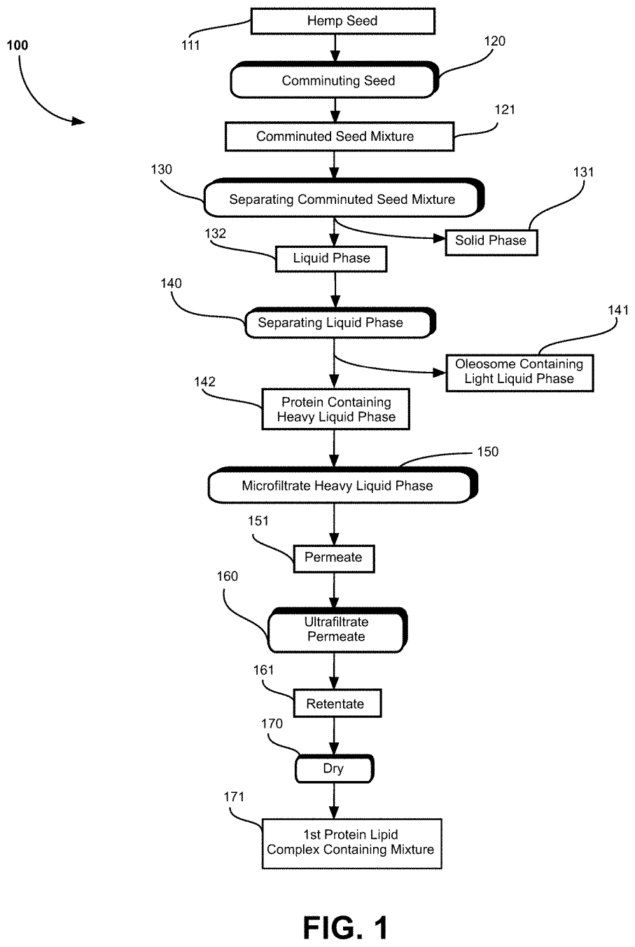

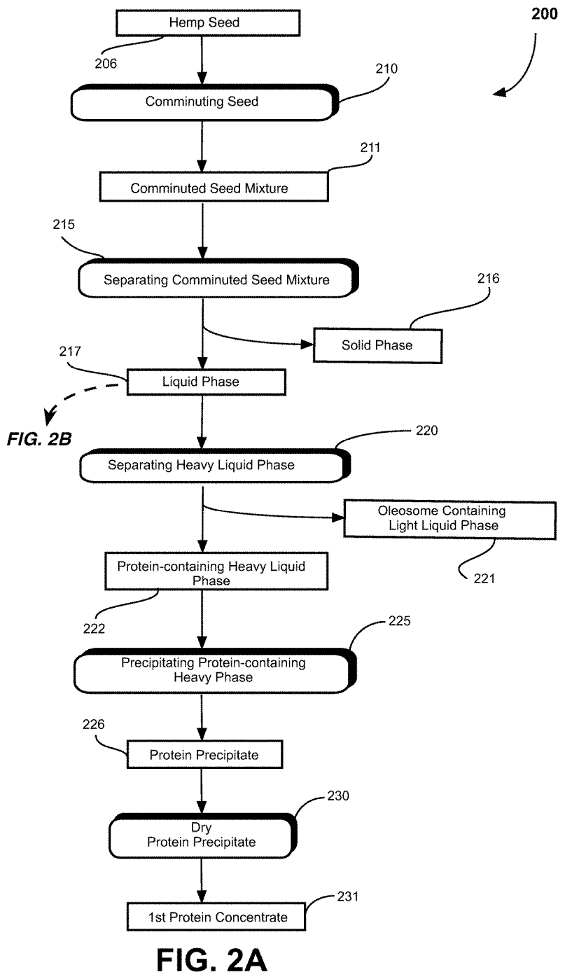

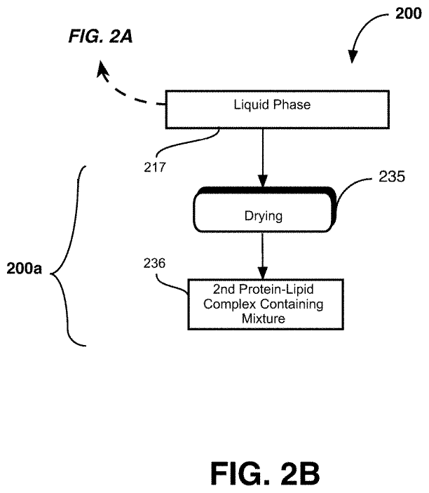

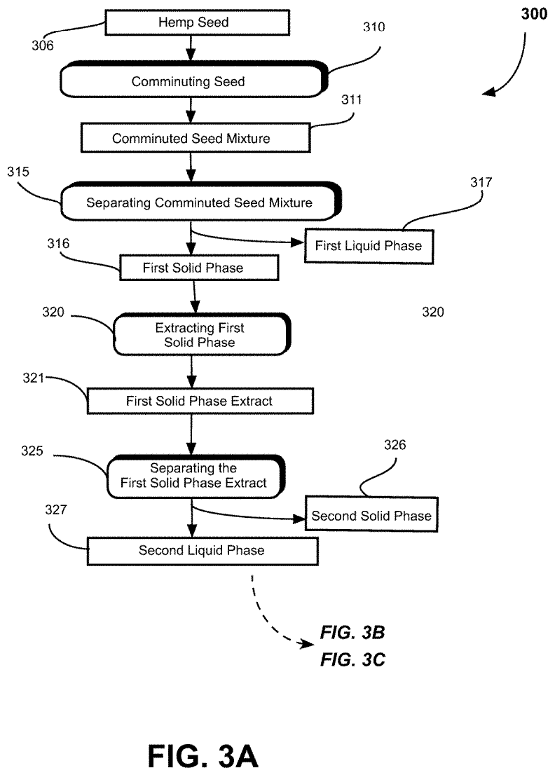

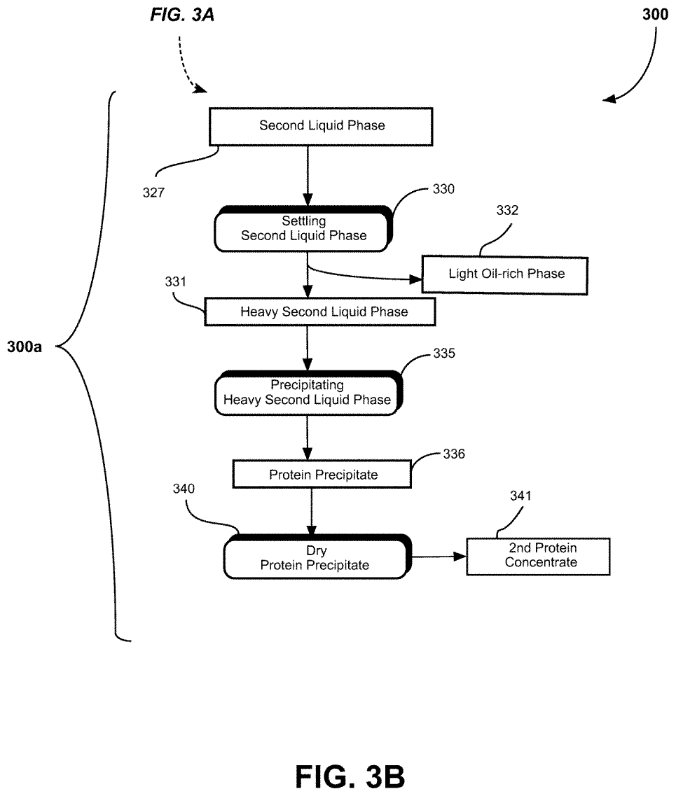

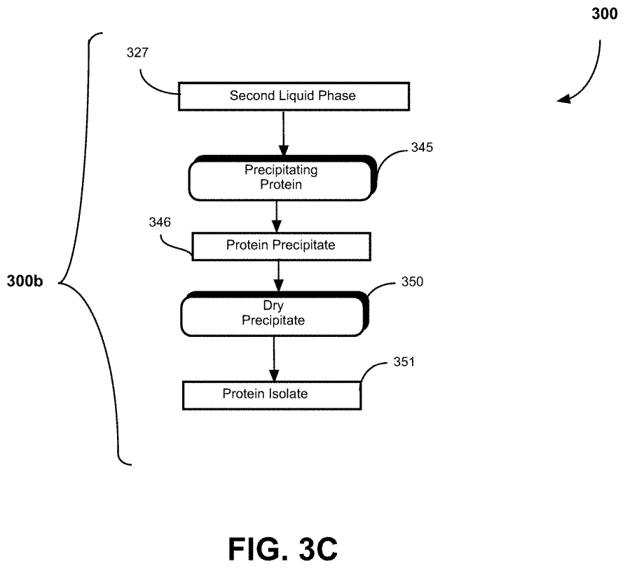

Various suitable techniques and methods for processing seed materials from hemp plant material to produce therefrom the present plant protein concentrates are disclosed in the following sections. FIG. 1, FIGS. 2A-2B, and FIGS. 3A-3C illustrate schematic diagrams of example processes 100, 200, and 300 wherein hemp seeds are selected and used as the starting plant materials for preparing hemp protein compositions in accordance with some embodiments of the present disclosure. Notably, process 100 illustrates a schematic diagram of a process for preparing protein-lipid complex containing mixtures, process 200 illustrates schematic diagrams for preparing protein concentrates and protein-lipid complex containing mixtures, and process 300 illustrates schematic diagrams for preparing protein concentrates and protein isolates

Thus, referring now to FIG. 1, example process 100 starts with providing a quantity of hemp seeds. Hemp seeds 111 may be obtained for example, by agriculturally producing and harvesting seed, and/or by purchasing seed from a commercial seed supplier. Prior to proceeding with the comminuting step 120, the hemp seeds 111 may optionally be screened and cleaned if necessary or so desired, to remove extraneous materials such as debris or non-intact seed material. Hemp seeds 111 may also optionally be washed or surface sterilized using, for example, a chemical agent such as bleach, to reduce contaminating biological agents such as bacteria or fungi that may be present on the seed coats. In some embodiments, undesirable seed parts may be removed for example, the seed coating, the hull, or the husk may be removed using for example, dehulling equipment with which those of skill in this art will generally be familiar. Furthermore, hemp seeds 111 may be soaked for a selected period of time for example, from about 30 minutes to about 24 hours, for example, for at least 6 hours in water.

Continuing in reference to FIG. 1, example process 100 further comprises comminuting step 120 wherein dehulled hemp seeds 111 are comminuted whereby is produced a comminuted mixture of seed particles 121, preferably having particle sizes in a range of between about 5 μm and about 200 μm. Comminuting step 120 generally reduces the seed volume as well as the particle size of the comminuted seed. Step 120 may be carried out by conveying of dehulled hemp seeds 111 into comminuting equipment such as a seed mill, a colloid mill, a hammer mill, a blade mill, a roller mill, and the like. In other embodiments, a homogenizer such as a high-pressure homogenizer, may be used to comminute of dehulled hemp seed 111. In yet other embodiments, a sequential combination of a mill and a homogenizer or other such equipment may be used to comminute the seed 111.

It should be further noted that the selection of the specific comminuting equipment and the operating conditions of the equipment may depend on the size of the selected hemp seed 111. However, regardless of the comminuting equipment that is selected, upon completion of step 120, the comminuted seed particle mixture 121 will have mean particle sizes in a range of from about 5 μm to about 200 μm or from about 5 μm to about 100 μm, or mean particle sizes in a range of about 10 μm, about 25 μm, about 50 μm, about 100 μm, about 125 μm, about 150 μm, about 175 μm, about 200 μm, and therebetween. Preferably, the comminution equipment and conditions are selected so that the comminuted hemp seed particles are homogenously sized, i.e. the particles can have tightly-centered mean-particle size, e.g. at least 90% of the particles can have a size not exceeding ±20% of the particle size, or not exceeding ±10% of the particle size, or not exceeding ±5% of the mean particle size. Furthermore, it should be noted that high temperatures i.e. temperatures in excess of 60° C. are avoided in the performance of comminuting step 121. Thus, comminuting step 121 may be conducted at ambient temperatures although it is understood that during operation of mechanical comminution equipment, the temperature of the seed mixture may increase above the ambient temperatures.

Comminuting step 121 may be carried out with the hemp seed 111 suspended in an aqueous solution. Examples of suitable aqueous solutions include water and dilute solutions comprising a sodium salt solution such as NaCl or Na₂SO₄. Examples of suitable dilute solutions may comprise about 50 mM of a sodium salt and/or about 50 mM of a strong acid. The aqueous solution may be added to the hemp seed 111 prior to conveyance into the comminuting equipment, or alternatively, while the hemp seed 111 is being discharged from a seed bin or other seed storage containers into the comminuting equipment. As hereinbefore noted, the use of organic solvents during performance of the comminuting step 121 is avoided.

Continuing in reference to FIG. 1, example process 100 may further comprise a separation step 130 during which the comminuted seed particle mixture is separated into a solid phase 131 and a liquid phase 132. Thus, separation step 130 yields two seed fractions. The separation step 130 may be carried out by conveying the comminuted seed particle mixture 121 into equipment suitable for separating the comminuted seed particle mixture 121 based on density differentials. Suitable separation equipment includes, for example, a centrifuge such as a two-phase decanter operated at modest gravitational forces to separate a light liquid phase 132 and a heavy solid phase 131 containing seed particle solids. The liquid phase 132 recovered after separation therefrom of the heavy solid phase 131, contains the majority of the seed oil, and the separated solid phase 131 contains solid seed particulate material including for example, seed hull particles.

The recovered solid phase 131 may have a dry-basis composition of about 8.3% (w/w) oil and 26.2% (w/w) protein (N×6.25).

Continuing in reference to FIG. 1, example process 100 may further comprise step 140 during which liquid phase 132 is separated into an oleosome containing light liquid phase 141 and a protein containing heavy liquid phase 142. Step 140 may be achieved by conveying the liquid phase 132 into separation equipment capable of further separating the liquid phase 132 based on density differential.

It should be noted that steps 130 and 140 may be performed concurrently by using a single-density differential-based separation equipment such as a 3-phase decanter capable of separating the comminuted seed particle mixture into a solid phase, a heavy liquid phase, and a light liquid phase.

Continuing in reference to FIG. 1, example process 100 may further comprise step 150 comprising subjecting the protein containing heavy liquid phase 142 to microfiltration treatment to obtain permeate 151. This may be achieved by using a membrane filter, for example, a membrane filter having a pore size of about 1 to about 3 μm.

Continuing in reference to FIG. 1, example process 100 may further comprise step 160 comprising subjecting permeate 151 to ultrafiltration treatment to obtain retentate 161. This may be achieved by a filter, for example, a filter having a molecular weight cut-off range from about 1 kDa to about 20 kDa.

Continuing in reference to FIG. 1, example process 100 may further comprise step 170 comprising drying retentate 161, to obtain first protein-lipid complex containing mixture concentrate 171. In general, the moisture content of the retentate 161 may be modulated by controlling the drying process, for example, by extending or decreasing the drying time and/or by increasing or decreasing the drying temperature. In this manner, a substantially dry first protein-lipid complex containing mixture 171 may be obtained. First protein-lipid complex containing mixture 171 may have from about 47% (w/w) to about 51% hemp protein (N×6.25), from about 32% (w/w) to about 36% (w/w) plant oil, and from about 3.4 (w/w) and to about 3.8% (w/w) carbohydrates. For example, the first protein-lipid complex containing mixture 171 may have a protein (N×6.25) content of 48.8% on an as-is basis (54.2% on a dry matter basis), 34.4% oil (on an as-is basis), 3.6% carbohydrate (on an as-is basis), and 3.3% ash (on an as-is basis). Furthermore, protein-lipid complex containing mixture 171 may have a moisture content of 9.9%. The total oil and protein (N×6.25) in protein-lipid complex containing mixture 171 may comprise a total of 83.2% (w/w) of the material, and may comprise a protein (N×6.25) to oil ratio of 1.4:1 on a weight-by-weight basis. Furthermore, protein-lipid complex containing mixture 171 may have an essential amino acid content of about 15% (on an as-is percentage basis), or about 30% (as a percentage of crude protein).

Example process 200 illustrated in FIGS. 2A-2B comprises another example embodiment. Thus, referring now to FIG. 2A, initially it is noted that initial steps and materials of process 200 correspond with initial steps and materials of process 100 (see: FIG. 1), notably, steps 210, 215 and 220 correspond with steps 120, 130 and 140, respectively, of process 100, and materials 206, 211, 216, 217, 221 and 222 correspond with materials 111, 121, 131, 132, 141 and 142, respectively, of process 100. Thus, these steps are in what follows not further discussed in reference to process 200, and the reader may refer back to the description of process 100 herein, or to Example 1, if desired. Instead, for the purposes of the following discussion of process 200, it will be assumed that protein containing heavy liquid phase 222 (142 in process 100) is available.

Thus, continuing in reference to FIG. 2, starting with protein containing heavy liquid phase 222, example process 200 may further comprise step 225 comprising separating the protein-containing heavy liquid phase 222 to recover therefrom a protein precipitate 226. Step 225 may be performed by acidifying the protein-containing heavy liquid phase 222 and adjusting the pH to a pH in the acidic pH range. Acid-precipitated protein 226 may be recovered using density-differential based separation equipment. Protein precipitate 226 then may be dried (step 230) to produce a first protein concentrate 231. First protein concentrate 231 may have from about 83% (w/w) to about 88% hemp protein (N×6.25), and from about 14% (w/w) to about 17% (w/w). For example, first protein concentrate 231 may have a protein content (N×6.25) of 86.6% (on an as-is basis), 14.7% plant oil (on an as-is basis), and 3.7% ash (on an as-is basis). Furthermore, first protein concentrate 231 may have a moisture content of 3.3%. For example, first protein concentrate 231 may have a protein content (N×6.25) of 84% (on an as-is basis), 15.5% plant oil (on an as-is basis), and 2.0% ash (on an as-is basis). Furthermore, first protein concentrate 231 may have a moisture content of 2.1%. Furthermore, the total oil and protein (N×6.25) in protein concentrate 231 may comprise a total 101.3% of the material, and a protein (N×6.25) to oil ratio of 5.9:1 on a weight-by-weight basis. Furthermore, protein concentrate 231 may have an essential amino acid content of between about 28% and about 29% (on an as-is percentage basis), or about 33% and about 34% (as a percentage of crude protein). Furthermore, the protein in protein concentrate 231 may be about 62% at pH 2, about 65% at pH 3, about 60% at pH 4, about 46% at pH 5, about 14% at pH 6, and about 1% at pH7. Furthermore, the oil-holding capacity (OHC) of protein concentrate 231 may be about 1 g oil/g of protein concentrate, and the water-hydration capacity (WHC) is about 1.6 g of water/g of protein concentrate. Furthermore, the emulsion stability of protein concentrate 231 may be about 51%. Furthermore, the foaming capacity of protein concentrate 231 may be about 15% and the foaming stability may be about 92%. Furthermore, protein concentrate 231 may have the following colorimetric characteristics: a lightness value L* of about 57%, a red/green tone value a* of about 2, and a yellow/blue tone value b* of about 15.

Referring next to FIG. 2B, process 200 may also include sub-process 200a. Sub-process 200a starts with liquid phase 217 (see also: FIG. 2A), and includes step 235 comprising drying liquid phase 217, using for example a spray drier, to obtain second protein-lipid complex containing mixture 236. Second protein-lipid complex containing mixture 236 may have from about 32% (w/w) to about 36% hemp protein (N×6.25), from about 58% (w/w) to about 62% (w/w) plant oil, and from about 0.5% (w/w) and to about 2.0% (w/w) carbohydrates. For example, second protein-lipid complex containing mixture 236 may have a protein content (N×6.25) of 33.6% on an as-is basis (34.7% on a dry matter basis), 59.3% oil (on an as-is basis), 1.2% carbohydrate (on an as-is basis), and 2.7% ash (on an as-is basis). Furthermore, second protein-lipid complex containing mixture 236 may have a moisture content of 3.2%. The total oil and protein (N×6.25) in protein-lipid complex-containing mixture 236 may comprise a total 92.9% of the material, and a protein (N×6.25) to oil ratio of 0.6:1. Furthermore, second protein-lipid complex-containing mixture 236 may have an oil-holding capacity OHC of about 1.24 g oil/g and a water-hydration capacity of about 0.59 g water/g. Furthermore, second protein-lipid complex containing mixture 236 may have a foaming capacity of about 198% and an emulsion stability at t=30 min of about 100% and at t=24 hrs of about 59%. Thus, starting with liquid phase 217 the performance of example sub-process 200a of example process 200 can yield protein-lipid complex containing mixture 236.

Example process 300 illustrated in FIGS. 3A-3C comprises another example embodiment. Thus, referring to FIGS. 3A-3C, it is noted that initial steps and materials of process 300 correspond with initial steps and materials of process 100 (see: FIG. 1), notably, steps 310 and 315 correspond with steps 120 and 130, respectively, of process 100, and materials 306, 311, 316 and 317 correspond with materials 111, 121, 131, and 132, respectively, of process 100. Thus, these steps are in what follows not further discussed in reference to process 300, and the reader may refer back to the description of process 100 herein, or to Example 1, if desired. Instead, for the purposes of the following discussion of process 300, it will be assumed that first solid phase 316 (131 in process 100) is available.

Thus, referring to FIGS. 3A-3B, starting with first solid phase 316, example process 300 may further comprise step 320 comprising liquefying solid phase 316. Solid phase 316, may, for example, have a moisture content of 50.8% and a dry-basis composition: 4.3% (w/w) oil, 32.1% (w/w) protein (N×6.25), 6.2% (w/w) ash and 57.4% (w/w) carbohydrate. Extraction step 320 may be conducted by mixing the first solid phase 316 with an aqueous solution. Thus, for example, 2 parts of water and 1 part of first solid phase 316 may be mixed to form a slurry. The slurry may be incubated for a period of time at a basic pH. In this manner, first solid extract 321 may be obtained. First solid extract 321 may be separated (step 325) into second solid phase 326 and second liquid phase 327, by subjecting first solid phase extract 325 to centrifugation. Second solid phase 326 may, for example, have a moisture content of 60.9% and a dry-basis composition: 1.6% (w/w) oil, 15.7% (w/w) protein (N×6.25), 6.9% (w/w) ash and 75.8% (w/w) carbohydrate. Second liquid phase 327 may, for example, have a moisture content of 95.7% and a dry-basis composition: 8.5% (w/w) oil, 90.7% (w/w) protein (N×6.25), 5.4% (w/w) ash and no detected carbohydrate.

Next, the second liquid phase may be processed according to a first example sub-process 300a of example process 300 shown in FIG. 3B or according to a second example sub-process 300b of example process 300 shown in FIG. 3C.

Referring, now to FIG. 3B, Next, step 330 of first example sub-process 300a of example process 300 may comprise settling second liquid phase 327 and obtaining a light oil-rich phase 332 and a heavy second liquid phase 331. This may be accomplished by gravitational settling, for example in a settling tank, wherein oil-rich phase 332 and heavy second liquid phase 331 may be formed. By gradual draining of the fluid from the bottom of the settling tank heavy second liquid phase 331 may be obtained. Next, heavy second liquid phase 331 may be precipitated (step 335) to obtain protein precipitate 336. This may be accomplished by acidifying the protein-containing heavy second liquid phase 331 and adjusting the pH to pH in the acidic pH range. The acid-precipitated protein may be recovered in the form of a protein precipitate 336 using density-differential based separation equipment. In order to obtain protein concentrate 341, protein precipitate 336 may be dried (step 340). Protein concentrate 341 may have a protein content (N×6.25) of, for example, 95% (on a dry matter basis) (from about 84% to about 87% protein on an as-is basis), from about 11% to about 18% plant oil (on a dry matter basis), from about 2% to about 3% ash (on a dry matter basis), and from about 3% to about 9% moisture. The total oil and protein (N×6.25) in protein concentrate 341 may comprise a total from about 97.9% to about 103% of the material, and may comprise a protein (N×6.25) to oil ratio of about 4.5:1 to about 7.9:1. Furthermore, protein concentrate 341 may have an essential amino acid content of about 26.5% to about 28% (on an as-is percentage basis), and about 31 to about 33% (as a percentage of crude protein). Furthermore, the percentage solubility of protein concentrate 341 may be between about 57% and about 62% at pH 2, between about 53% and about 64% at pH 3, between 49% and about 51% at pH 4, between about 39% and about 46% at pH 5, between about 1% and about 2% at pH 6, and between about 30% and 35% at pH 7. Furthermore, the oil-holding capacity of protein concentrate 341 may be from about 0.8 to about 1.2 g oil/g of protein, and the water-hydration capacity may be from about 1.5 to about 1.8 g water/g of protein. Furthermore, the emulsion stability of protein concentrate 341 may be between 53% and 56%. Furthermore, the foaming capacity of protein concentrate 341 may be about 15% and the foaming stability may range from about 92% to about 100%. Furthermore, protein concentrate 341 may have the following colorimetric characteristics: a lightness value L* of between about 60% and about 63%, a red/green tone value a* of between about 0.7 and about 0.8, and a yellow/blue tone value b* of between about 9 and about 13.

Thus, starting with liquid phase 327, the performance of example sub-process 300a of example process 300 may yield second protein concentrate 341.

Referring next to FIG. 3C, shown therein is a second example sub-process 300b of example process 300. Subprocess 300b starts again with second liquid phase 327. Example sub-process 300b may further comprise step 345 comprising precipitating second liquid phase 327 to obtain protein precipitate 346. This may be achieved by acidifying second liquid phase 327 and adjusting the pH to a pH in the acidic pH range. The acid-precipitated protein may be recovered therefrom in the form of a protein precipitate 346 using density differential-based separation equipment. In order to obtain protein isolate 351, protein precipitate 346 may be dried (step 350). Protein isolate 351 may, for example, have a protein content (N×6.25) of, for example, from about 94% (w/w) to about 98% (w/w), and from about 5% to about 8% of plant oil. Protein isolate 351 may, for example, have a protein content (N×6.25) of, for example, 95.5% (on a dry matter basis) (93.1% protein on an as-is basis), 6.6% plant oil (on an as-is basis), 4.3% ash (on an as-is basis), and 2.6% moisture. The total oil and protein (N×6.25) in protein isolate 351 may comprise a total 99.62% of the material, in a protein (N×6.25) to oil ratio of 14.2:1 Thus, starting with liquid phase 327, the performance of example sub-process 300b of example process 300 may yield protein isolate 351.

To briefly recap, the example processes 100, 200, and 300 may each provide selectable protein precipitates which may be selectively dried to form prepared protein concentrates. The prepared hemp protein concentrates may comprise from about 30% (w/w) to about 95% hemp protein (N×6.25), from about 5% (w/w) and to about 60% (w/w) plant oil.

The hemp protein compositions, i.e. the hemp protein concentrates, the hemp protein isolates and the hemp protein-lipid complex containing mixtures, of the present disclosure may be prepared without exposing the starting selected hemp plant materials to high temperatures or to solvents. Thus, the proteins from hemp plants recovered by the fractionation processes described herein will not have sustained any heat damage or solvent damage. As a result, hemp protein compositions disclosed herein may be light colored, relatively odorless, and bland in taste. Furthermore, the hemp proteins within the various hemp protein compositions described herein may be substantially non-denatured and may retain their primary and/or their secondary and/or their tertiary three-dimensional structures.

According to another aspect, the hemp protein concentrates may comprise at least about 84% (w/w) hemp protein (N×6.25), and from about 11% (w/w) to about 16% (w/w) plant oil.

According to one aspect, the hemp protein concentrate may comprise at least 85% (w/w) and less than 90% (w/w) hemp proteins (N×6.25).

According to one aspect, the hemp protein concentrate may comprise least 86% (w/w) and less than 90% (w/w) hemp proteins (N×6.25).

According to another aspect, the hemp protein isolates may comprise at least 90% (w/w) hemp protein (N×6.25), and from about 6% (w/w) to about 8% (w/w) plant oil.

According to another aspect, the hemp protein isolates may comprise at least 91% (w/w) hemp protein (N×6.25).

According to another aspect, the hemp protein isolates may comprise at least 92% (w/w) hemp protein (N×6.25).

According to another aspect, the hemp protein isolates may comprise at least 93% (w/w) hemp protein (N×6.25).

According to another aspect, the hemp protein-lipid complex containing mixtures may comprise from about 34% (w/w) to about 49% (w/w) protein (N×6.25), from about 34% (w/w) to about 59% (w/w) plant oil, and from about 1% (w/w) to about 4% (w/w) carbohydrates.

According to another aspect, hemp protein concentrates may comprise a total protein (N×6.25) and plant oil content of about 100% (w/w).

According to another aspect, the hemp protein isolates may comprise a total protein (N×6.25) and plant oil content of about 100% (w/w).

According to another aspect, the hemp protein-lipid complex containing mixture may comprise a total protein (N×6.25) and plant oil content of at least about 83% (w/w).

According to another aspect, the hemp protein concentrate may comprise a ratio of protein (N×6.25) to oil is at least 5.3 on a weight-by-weight basis.

According to another aspect, the hemp protein isolate, may comprise a ratio of protein (N×6.25) to oil is at least 14:1 on a weight-by-weight basis.

According to another aspect, the hemp protein-lipid complex containing mixture may comprise a protein (N×6.25) to oil ratio of at least about 0.6:1 on a weight-by-weight basis.

Another embodiment of the present disclosure relates to hemp protein compositions comprising weight percentages of essential amino acids of at least about 30% by weight crude protein.

Another embodiment of the present disclosure relates to hemp protein compositions comprising weight percentages of essential amino acids of at least about 35% by weight crude protein.

According to an aspect, the weight percentages of lysine in the hemp protein compositions may be at least about 2.8% by weight crude protein.

According to another aspect, the weight percentages of lysine in the hemp protein compositions may be at least about 4.9% by weight crude protein.

According to some embodiments of the present disclosure, hemp protein compositions produced by the processes described herein may be used as ingredients in nutritional formulations. In order to prepare the nutritional formulations, one or more of the protein compositions from selected hemp species disclosed herein may be contacted with or blended with or mixed together with at least one other formulary ingredient suitable for use to prepare a nutritional product composition. Furthermore, at least one other formulary ingredient may be provided in any suitable form such as for example, a solution, a suspension, a gel, a liquid, a solid, a powder, a crystal, and the like. The quantity of the at least one other formulary ingredient may vary and may depend on the type of nutritional formulation that is being prepared. Furthermore, a plurality of additional formulary ingredients may be provided, for example 2, 3, 4, 5, 6, 7, 8, 9, 10 or more formulary ingredients to prepare the nutritional formulation.

In some embodiments, a formulation suitable for inclusion in a nutritional product comprising a mixture of formulary ingredients may be pre-formed, and the protein composition may be separately provided and incorporated into the pre-formed formulary ingredient mixture.

In some embodiments, the hemp protein composition may be incorporated during preparation of the nutritional formulation. In such embodiments, the hemp protein composition may be added separately or alternatively, the hemp protein composition may be incorporated together with one or more other formulary compounds.

The final concentration of the hemp protein composition in the nutritional product may vary. In some embodiments, the hemp protein composition may comprise at least about 10% (w/w) of the nutritional formulation. In other embodiments, the hemp protein composition may comprise at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the nutritional formulation. The concentration of the hemp protein composition may be optimized or adjusted by preparing a plurality of sample nutritional formulations, wherein each formulation comprises a different concentration of the hemp protein composition, then evaluating each of the formulations with reference to one or more nutritional effects, then selecting one or more of the formulations to provide a selected desirable effect.

In some embodiments, the additional formulary ingredient incorporated in the nutritional formulations of the present disclosure may be a natural ingredient. Since the protein compositions disclosed herein are natural compositions, in some embodiments, the nutritional formulations may be formulated using additional natural formulary ingredients thereby providing one or more natural nutritional formulations.

In some embodiments, the additional formulary ingredient may be a synthetic ingredient, for example stabilizing agents such as xanthan and gellan, coagulating agents such as calcium sulfate and magnesium chloride, and agglomerating agents such as hydrolysed lecithin for example.

In accordance with the foregoing, the present disclosure provides another embodiment relating to methods for preparing nutritional formulations comprising the plant protein compositions disclosed herein, wherein the methods comprise:
(i) providing a plant protein composition from a selected hemp species comprising from about 30% (w/w) to about 95% hemp protein (N×6.25), from about 5% (w/w) and to about 60% (w/w) plant oil, the balance of the protein composition substantially being constituted by carbohydrates, water and ash;
(ii) providing one or more formulary ingredients suitable for inclusion into a nutritional formulation; and
(iii) formulating the plant protein composition with the formulary ingredient by blending or mixing to produce a nutritional formulation comprising the plant protein composition.

Another embodiment of the present disclosure relates to use of a hemp protein composition disclosed herein as an ingredient for preparing a nutritional formulation, wherein the hemp protein composition comprises from about 30% (w/w) to about 95% hemp protein, from about 5% (w/w) and to about 60% (w/w) plant oil, the balance of the hemp protein composition substantially being constituted by carbohydrates, water, and ash.

Another embodiment of the present disclosure relates to use of a hemp protein composition disclosed herein as an ingredient for preparing a nutritional formulation, wherein the hemp protein composition comprises from about 30% (w/w) to about 95% hemp protein, from about 5% (w/w) and to about 60% (w/w) plant oil, up to about 4% (w/w) carbohydrates, the balance of the hemp protein composition substantially being constituted by water and ash.

Another embodiment of the present disclosure relates to use of a hemp protein composition disclosed herein as an ingredient for preparing a nutritional formulation, wherein the hemp protein composition comprises from about 30% (w/w) to about 95% hemp protein, from about 5% (w/w) to about 60% (w/w) plant oil, and from about 1% (w/w) to about 4% (w/w) carbohydrates, the balance of the hemp protein composition substantially being constituted by water and ash.

Another embodiment of the present disclosure relates to a nutritional formulation comprising a hemp protein composition comprising at least about 65% (w/w) or more plant protein, from about 30% (w/w) to about 95% hemp protein (N×6.25), from about 5% (w/w) to about 60% (w/w) plant oil, the balance of the hemp protein composition substantially being constituted by carbohydrates, water and ash.

Another embodiment of the present disclosure relates to a nutritional formulation comprising a hemp protein composition comprising at least about 65% (w/w) or more plant protein, from about 30% (w/w) to about 95% hemp protein (N×6.25), from about 5% (w/w) to about 60% (w/w) plant oil, and up to about 4% (w/w) carbohydrates, the balance of the hemp protein composition substantially being constituted by water and ash.

Another embodiment of the present disclosure relates to a nutritional formulation comprising a hemp protein composition comprising at least about 65% (w/w) or more plant protein, from about 30% (w/w) to about 95% hemp protein (N×6.25), from about 5% (w/w) to about 60% (w/w) plant oil, and from about 1% (w/w) to about 4% (w/w) carbohydrates, the balance of the hemp protein composition substantially being constituted by water and ash.

Some nutritional formulations incorporating the hemp protein compositions disclosed herein may be suitable for use as an animal feed. Some nutritional formulations incorporating the hemp protein compositions disclosed herein may be suitable for use as a fish feed or an aquaculture feed. Some nutritional formulations incorporating the hemp protein compositions disclosed herein may be suitable for human consumption. Some nutritional formulations incorporating the hemp protein compositions disclosed herein may be suitable for use as a poultry feed or a pig feed or a companion animal feed or a feed formulation for juvenile animals.

### EXAMPLES

Hereinafter are provided examples of further specific embodiments for performing the methods of the present disclosure, as well as embodiments representing the compositions of the present disclosure.

### Example 1—Preparation of a Protein-Lipid Complex Containing Mixture

This example refers to process 100 depicted in FIG. 1. Cannabis sativa seeds 111 (32.5% oil content, 25.6% protein) fed into a mill, along with a bicarbonate buffer in reverse osmosis (RO) water to produce a wet-milled aqueous slurry of seed. The slurry underwent immediate particle size reduction through a mill. The homogenized slurry (i.e. comminuted seed mixture 121) was immediately fed into a centrifuge decanter with sufficient force on the slurry to enable separation of solid phase 131 and liquid phase 132. Solid phase 131 was the first co-product of process 100 and had a dry-basis oil and protein contents of 8.3% (w/w) and 26.2% (w/w), respectively. The corresponding decanter liquid phase 132 (399 kg), comprising a mixture of liquefied seed components was separated in a two-phase disc-stack separator. The resulting oleosome-containing light liquid phase 141 comprises most of the seed oil (>75%) whereas the corresponding heavy liquid phase 142 has the following dry-basis oil and protein contents: 25.3% (w/w) and 18.5% (w/w) respectively. Heavy liquid phase 142 (276 kg) underwent microfiltration and the resultant permeate 151 underwent ultrafiltration to produce retentate 161, a sample of which was dried to produce a first protein-lipid complex containing mixture 171 from example process 100 with the composition shown in Table 1, an amino acid composition shown in Table 2, and a fatty acid composition shown in Table 3.

TABLE 1
First protein-lipid complex containing mixture: 171

| Composition: | |
|---|---|
| Crude Protein (% dry matter) (N × 6.25) | 54.2 |
| Crude Protein (%) | 48.8 |
| Moisture (%) | 9.9 |
| Crude Fat (%) | 34.4 |
| Crude Ash (%) | 3.3 |
| Carbohydrate (%, by difference) | 3.6 |

The total oil and protein in protein-lipid complex containing mixture 171 comprised a total of 83.2% (w/w) of the material, in a protein to oil ratio of 1.4:1.

TABLE 2
First protein-lipid complex containing mixture 171.

| | % As-is | % of Crude |
|---|---|---|
| Amino Acids: | basis | Protein |
| Alanine | 1.75 | 3.59 |
| Arginine | 7.82 | 16.02 |
| Aspartic acid | 4.63 | 9.49 |
| Glutamic acid | 9.39 | 19.24 |
| Glycine | 2.39 | 4.90 |
| Histidine | 1.74 | 3.57 |
| Isoleucine | 1.36 | 2.79 |
| Leucine | 2.31 | 4.73 |
| Phenylalanine | 1.55 | 3.18 |
| Proline | 2.86 | 5.86 |
| Serine | 3.02 | 6.19 |
| Threonine | 2.25 | 4.61 |
| Lysine | 2.38 | 4.88 |
| Tyrosine | 1.54 | 3.16 |
| Valine | 1.87 | 3.83 |
| Cysteine | 1.00 | 2.05 |
| Methionine | 0.91 | 1.86 |
| Tryptophan | 0.49 | 1.00 |
| Essential Amino acids | 14.86 | 30.45 |

TABLE 3
First protein-lipid complex containing mixture 171.

| | % As-is |
|---|---|
| Fatty Acids: | basis |
| C18:1n9 Oleic | 11.86 |
| C18:1 Octadecenoic | 0.92 |
| C18:2 Linoleic | 54.32 |
| C18:3n6 gamma-Linolenic | 3.41 |
| C18:3n3 alpha-Linolenic | 14.28 |
| C18:4 Octadecatetraenoic | 0.99 |
| C20 Arachidic | 0.95 |
| C20:1 Eicosenoic | 0.49 |
| C20:2n6 Eicosadienoic | 0.07 |
| C22 Behenic | 0.43 |
| C22:1n9 Erucic | 0.03 |
| C24 Lignoceric | 0.22 |
| Others | 0.71 |
| Total Saturates | 12.75 |
| Total Monounsaturates | 13.47 |
| Total Polyunsaturates | 73.07 |
| Total Omega 3 | 15.27 |
| Total Omega 6 | 57.80 |
| Total Omega 9 | 12.38 |

### Example 2—Preparation of a Hemp Protein Concentrate

This example refers to process 200 depicted in FIG. 2. Clean (<1% dockage) Cannabis Sativa L. seeds 206 (29.3% oil content, 24.9% protein content) were washed. The clean seeds were fed by an auger hammer mill and combined with reverse osmosis water to produce a wet-milled aqueous slurry of seed. The slurry underwent immediate particle size reduction by processing through a mill. The pH of the final homogenized slurry (i.e. comminuted seed mixture 211) was adjusted to 10, which was then fed into a centrifuge decanter with sufficient force on the slurry to enable separation of solid phase 216 and liquid phase 217. Solid phase 216 is the first co-product of process 200 and the corresponding decanter liquid phase 217 (671.9 kg), comprising a mixture of liquefied seed components, was separated in a separator. The resulting oleosome-containing light liquid phase 221 serves to direct the majority of the seed oil (>65%) away from the production pathway for protein in order to facilitate low-fat protein concentrates. To that end, the pH of corresponding heavy liquid phase 222 (515.5 kg) was adjusted to 5.5 and the resulting mixture was separated in a clarifier centrifuge. A sample of resulting pellet precipitate 226 (84.7 kg) was dried to produce a first protein concentrate 231 from example process 200 with the composition shown in Table 4 and an amino acid composition shown in Table 5.

TABLE 4
First protein concentrate: 231

| Composition: | |
|---|---|
| Crude Protein (%) (N × 6.25) | 86.6 |
| Moisture (%) | 3.3 |
| Crude Fat (%) | 14.7 |
| Crude Ash (%) | 3.7 |
| Carbohydrate (by difference) | — |

The total oil and protein in protein concentrate 231 comprised a total 101.3% of the material, in a protein to oil ratio of 5.9:1.

TABLE 5
First protein concentrate: 231

| | % As-is | % of Crude |
|---|---|---|
| Amino Acids: | basis | Protein |
| Alanine | 3.69 | 4.26 |
| Arginine | 10.99 | 12.70 |
| Aspartic acid | 8.97 | 10.36 |
| Glutamic acid | 14.57 | 16.83 |
| Glycine | 3.55 | 4.10 |
| Histidine | 2.27 | 2.62 |
| Isoleucine | 3.64 | 4.21 |
| Leucine | 5.79 | 6.69 |
| Phenylalanine | 3.96 | 4.57 |
| Proline | 3.09 | 3.57 |
| Serine | 4.17 | 4.82 |
| Threonine | 2.9 | 3.35 |
| Lysine | 2.85 | 3.29 |
| Tyrosine | 3.06 | 3.54 |
| Valine | 4.48 | 5.18 |
| Cysteine | 1.1 | 1.27 |
| Methionine | 1.92 | 2.22 |
| Tryptophan | 1.08 | 1.25 |
| Essential | 28.89 | 33.38 |
| Amino Acids | | |

### Example 3—Preparation of Another Hemp Protein Concentrate

This example refers to process 200 depicted in FIG. 2. Clean (<1% dockage) Cannabis Sativa L. seeds 206 (29.3% oil content, 24.9% protein content) were washed. The clean seeds were fed into an auger mill and combined with reverse osmosis water to produce a wet-milled aqueous slurry of seed. The slurry underwent immediate particle size reduction by processing through the mill. The pH of the final homogenized slurry (i.e. comminuted seed mixture 211) was adjusted to 10 and the resulting slurry was fed into a centrifuge decanter with sufficient force on the slurry to enable separation of solid phase 216 and liquid phase 217. Solid phase 216 was the first co-product of process 200 and had a moisture content of 54.8% and the following dry-basis composition: 6.8% (w/w) oil, 15.0% (w/w) protein and 23.5% (w/w) ash and carbohydrate (calculated by difference). The corresponding decanter liquid phase 217 comprising a mixture of liquefied seed components, was separated in a separator. The resulting oleosome-containing light liquid phase 221 (236.1 kg) served to direct the majority of the seed oil (>65%) away from the production pathway for protein in order to facilitate low-fat protein concentrates. The corresponding heavy liquid phase 222 had the following dry-basis composition: 14.9% (w/w) oil, 70.9% (w/w) protein and 14.3% (d/w) of ash and carbohydrate (Calculated by difference). The pH of heavy liquid phase 222 was adjusted to 5.5 and the resulting mixture was separated in a clarifier centrifuge. A sample of resulting pellet precipitate 226 was dried to give a first protein concentrate 231 from example process 200 with the composition shown in Table 6 and an amino acid composition shown in Table 7.

TABLE 6
First protein concentrate: 231

| Composition: | |
|---|---|
| Crude Protein (% as-is) (N × 6.25) | 84.0 |
| Moisture (%) | 2.1 |
| Crude Fat (%) | 15.5 |
| Crude Ash (%) | 2.0 |
| Carbohydrate (by difference) | — |

The total oil and protein in first protein concentrate 231 comprised a total 99.5% of the material, in a protein to oil ratio of 5.4:1.

TABLE 7
First protein concentrate: 231

| | % As-is | % of Crude |
|---|---|---|
| Amino Acids: | basis | Protein |
| Alanine | 3.61 | 4.30 |
| Arginine | 11.06 | 13.17 |
| Aspartic acid | 8.9 | 10.60 |
| Glutamic acid | 14.52 | 17.29 |
| Glycine | 3.44 | 4.10 |
| Histidine | 2.26 | 2.69 |
| Isoleucine | 3.57 | 4.25 |
| Leucine | 5.66 | 6.74 |
| Phenylalanine | 3.93 | 4.68 |
| Proline | 3.1 | 3.69 |
| Serine | 4.12 | 4.90 |
| Threonine | 2.84 | 3.38 |
| Lysine | 2.84 | 3.38 |
| Tyrosine | 3.03 | 3.61 |
| Valine | 4.42 | 5.26 |
| Cysteine | 1 | 1.19 |
| Methionine | 1.86 | 2.21 |
| Tryptophan | 1.1 | 1.31 |
| Essential Amino Acids | 28.48 | 33.90 |

### Example 4—Preparation of Another Hemp Protein Concentrate

This example refers to process 300 and sub-process 300a depicted in FIG. 3A and FIG. 3B. Cannabis sativa seeds 306 (29.3% oil content, 24.9% protein) were washed. The washed seeds were fed by an auger to a mill along with reverse osmosis (RO) water to produce a wet-milled aqueous slurry of seed.

The resulting slurry underwent immediate and continuous particle size reduction by processing through homogenizers. The homogenized slurry (i.e. comminuted seed mixture 311) was immediately fed into a centrifuge with sufficient force on the slurry to enable separation of solid phase 316 and liquid phase 317. Solid phase 316 had a moisture content of 50.8% and a dry-basis composition: 4.3% (w/w) oil, 32.1% (w/w) protein, 6.2% (w/w) ash and 57.4% (w/w) carbohydrate. The corresponding decanter liquid phase 317 served to direct the majority of the seed oil (>80%) away from the protein production pathway. The solid phase 316 was combined with RO-water in a holding tank while maintaining constant mixing of the contents. The pH of the solid phase slurry was adjusted to 10. Following extraction, the solid phase extract 321 was fed into a centrifuge decanter with sufficient force on the solid phase extract 321 to separate a second solid phase 326 and a second liquid phase 327. The second solid phase 326 had a moisture content of 60.9% and a dry-basis composition: 1.6% (w/w) oil, 15.7% (w/w) protein, 6.9% (w/w) ash and 75.8% (w/w) carbohydrate. The second liquid phase 327 (217.0 kg) had a moisture content of 95.7% and a dry-basis composition: 8.5% (w/w) oil, 90.7% (w/w) protein, 5.4% (w/w) ash and no detected carbohydrate.

The second liquid phase 327 was then allowed to stand and settle under gravity in a tank for 82 mins, to allow for any oil-rich phase to separate out to the top. The bottom 75% of the tank contents were drained and collected to produce the heavy second liquid phase 331. The pH of the heavy second liquid phase 331 was adjusted to 5.0 and the resulting mixture was separated in a clarifier centrifuge. A sample of the resulting pellet or protein precipitate 336 was dried to produce a second protein concentrate 341 from example process 300 and subprocess 300a with the composition shown in Table 8 and an amino acid composition shown in Table 9. The total oil and protein in second protein concentrate 341 comprised a total 97.9% of the material, in a protein to oil ratio of 7.9:1.

TABLE 8
Second protein concentrate: 341

| Composition: | |
|---|---|
| Crude Protein (% as-is) (N × 6.25) | 86.94 |
| Moisture (%) | 9.00 |
| Crude Fat (%) | 10.99 |
| Crude Ash (%) | 3.07 |
| Carbohydrate (by difference, %) | — |

TABLE 9
Second protein concentrate: 341

| | % As-is | % of Crude |
|---|---|---|
| Amino Acids: | basis | Protein |
| Alanine | 3.57 | 4.11 |
| Arginine | 11.75 | 13.52 |
| Aspartic acid | 9.35 | 10.75 |
| Glutamic acid | 14.97 | 17.22 |
| Glycine | 3.47 | 3.99 |
| Histidine | 2.25 | 2.59 |
| Isoleucine | 3.34 | 3.84 |
| Leucine | 5.58 | 6.42 |
| Phenylalanine | 3.96 | 4.55 |
| Proline | 3.11 | 3.58 |
| Serine | 4.36 | 5.01 |
| Threonine | 2.83 | 3.26 |
| Lysine | 2.40 | 2.76 |
| Tyrosine | 3.08 | 3.54 |
| Valine | 4.10 | 4.72 |
| Cysteine | 0.95 | 1.09 |
| Methionine | 1.81 | 2.08 |
| Tryptophan | 1.16 | 1.33 |
| Essential Amino Acids | 27.43 | 31.55 |

### Example 5—Preparation of Another Hemp Protein Concentrate

This example refers to process 300 and subprocess 300b depicted in FIG. 3A and FIG. 3C. Cannabis sativa seeds 306 (33.3% oil content, 24.3% protein) were washed. The washed seeds were fed by an auger to a mill and combined with reverse osmosis (RO) water to produce a wet-milled aqueous slurry of seed. The resulting slurry underwent immediate and continuous particle size reduction by processing with homogenizers. The homogenized slurry (i.e. comminuted seed mixture 311) was immediately fed into a centrifuge decanter with sufficient force to enable separation of solid phase 316 and liquid phase 317. Solid phase 316 had a moisture content of 45.7% and a dry-basis composition: 7.2% (w/w) oil, 37.3% (w/w) protein, 7.2% (w/w) ash and 49.3% (w/w) carbohydrate. The corresponding decanter liquid phase 317, comprising a mixture of liquefied seed components, served to direct the majority of the seed oil (>85%) away from the protein production pathway. The solid phase 320 was combined in a holding tank while maintaining constant mixing of the contents. The pH of the solid phase slurry was adjusted to 10.5. Following extraction, the solid phase extract 321 was fed into a centrifuge decanter with sufficient force on the solid phase extract 321 to separate a second solid phase 326 and a second liquid phase 327. Separation of the slurry in the decanter occurred in 40 minutes. The second solid phase 326 (29.6 kg) had a moisture content of 62.5% and a dry-basis composition: 5.3% (w/w) oil, 14.7% (w/w) protein, 7.1% (w/w) ash and 72.9% (w/w) carbohydrate. The second liquid phase 327 (166 kg) had a moisture content of 96.8% and a dry-basis composition: 11.6% (w/w) oil, 85.9% (w/w) protein, 0.6% (w/w) ash and 1.9% (w/w) carbohydrate.

The pH of the second liquid phase 327 was reduced and the resulting mixture was separated in a clarifier centrifuge. A sample of the resulting pellet or protein precipitate 346 was dried to give a second protein concentrate 341 from example process 300 and subprocess 300b with the composition shown in Table 10.

TABLE 10
Second protein concentrate: 341

| Composition: | |
|---|---|
| Crude Protein (% dry matter) (N × 6.25) | 87.6 |
| Crude Protein (% as-is) (N × 6.25) | 84.4 |
| Moisture (%) | 3.6 |
| Crude Fat (%) | 16.8 |
| Crude Ash (%) | 2.2 |
| Carbohydrate (by difference, %) | — |

The total oil and protein in second protein concentrate 341 comprised a total 101.2% of the material, in a protein to oil ratio of 5.0:1.

TABLE 11
Second protein concentrate: 341

| | % As-is | % of Crude |
|---|---|---|
| Amino Acids: | basis | Protein |
| Alanine | 3.37 | 3.99 |
| Arginine | 11.01 | 13.05 |
| Aspartic acid | 8.73 | 10.34 |
| Glutamic acid | 14.17 | 16.79 |
| Glycine | 3.32 | 3.93 |
| Histidine | 2.16 | 2.56 |
| Isoleucine | 3.39 | 4.02 |
| Leucine | 5.39 | 6.39 |
| Phenylalanine | 3.76 | 4.45 |
| Proline | 2.88 | 3.41 |
| Serine | 4.04 | 4.79 |
| Threonine | 2.63 | 3.12 |
| Lysine | 2.37 | 2.81 |
| Tyrosine | 2.94 | 3.48 |
| Valine | 4.08 | 4.83 |
| Cysteine | 1.01 | 1.20 |
| Methionine | 1.88 | 2.23 |
| Tryptophan | 1.04 | 1.23 |
| Essential Amino Acids | 26.7 | 31.64 |

### Example 6—Preparation of Another Hemp Protein Concentrate

This example refers to process 300 and subprocess 300b depicted in FIG. 3A and FIG. 3C. Cannabis sativa seeds 306 (33.3% oil content, 24.3% protein) were washed. The washed seeds were fed by an auger to a mill and combined with reverse osmosis (RO) water to produce a wet-milled aqueous slurry of seed. The resulting slurry underwent immediate and continuous particle size reduction by processing with homogenizers. The homogenized slurry (i.e. comminuted seed mixture 311) was immediately fed into a centrifuge decanter with sufficient force to enable separation of solid phase 316 and liquid phase 317. Solid phase 316 had a moisture content of 47.7% and a dry-basis composition: 8.1% (w/w) oil, 36.1% (w/w) protein, 6.1% (w/w) ash and 49.3% (w/w) carbohydrate. The corresponding decanter liquid phase 317 comprising a mixture of liquefied seed components, served to direct the majority of the seed oil (>85%) away from the protein production pathway. The solid phase 320 was combined with RO-water in a holding tank while maintaining constant mixing of the contents. The pH of the solid phase slurry was adjusted to 10.5. Following extraction, the solid phase extract 321 was fed into a centrifuge decanter with sufficient force on the solid phase extract 321 to separate a second solid phase 326 and a second liquid phase 327. Separation of the slurry in the decanter occurred in 36 minutes. The second solid phase 326 (42.0 kg) had a moisture content of 57.8% and a dry-basis composition: 4.8% (w/w) oil, 17.5% (w/w) protein, 7.2% (w/w) ash and 70.5% (w/w) carbohydrate. The second liquid phase 327 (114 kg) had a moisture content of 96.8% and a dry-basis composition: 23.5% (w/w) oil, 79.1% (w/w) protein, 3.4% (w/w) ash, and no detected carbohydrate.

The pH of the second liquid phase 327 was reduced to 5.0 and the resulting mixture was separated in a clarifier centrifuge. A sample of the resulting pellet or protein precipitate 346 was dried to produce a second protein concentrate 341 from example process 300 and subprocess 300b with the composition shown in Table 12.

TABLE 12
Second protein concentrate: 341

| Composition: | |
|---|---|
| Crude Protein (% dry matter) (N × 6.25) | 87.2 |
| Crude Protein (% as-is) (N × 6.25) | 84.6 |
| Moisture (%) | 3.0 |
| Crude Fat (%) | 18.1 |
| Crude Ash (%) | 1.9 |
| Carbohydrate (by difference, %) | — |

The total oil and protein in second protein concentrate 341 comprised a total 102.7% of the material, in a protein to oil ratio of 4.7:1.

TABLE 13
Second protein concentrate: 341

| | % As-is | % of Crude |
|---|---|---|
| Amino Acids: | basis | Protein |
| Alanine | 3.49 | 4.13 |
| Arginine | 11.34 | 13.40 |
| Aspartic acid | 9.02 | 10.66 |
| Glutamic acid | 14.69 | 17.36 |
| Glycine | 3.44 | 4.07 |
| Histidine | 2.23 | 2.64 |
| Isoleucine | 3.51 | 4.15 |
| Leucine | 5.59 | 6.61 |
| Phenylalanine | 3.9 | 4.61 |
| Proline | 3.01 | 3.56 |
| Serine | 4.22 | 4.99 |
| Threonine | 2.74 | 3.24 |
| Lysine | 2.41 | 2.85 |
| Tyrosine | 3.03 | 3.58 |
| Valine | 4.19 | 4.95 |
| Cysteine | 1.01 | 1.19 |
| Methionine | 1.92 | 2.27 |
| Tryptophan | 1.05 | 1.24 |
| Essential Amino Acids | 27.54 | 32.55 |

### Example 7—Preparation of a Hemp Protein Isolate

This example refers to process 300 and subprocess 300b depicted in FIG. 3A and FIG. 3C. Cannabis sativa seeds 306 (29.3% oil content, 24.9% protein) were washed. The washed seeds were fed by an auger to a mill and combined with reverse osmosis water to produce a wet-milled aqueous slurry of seed. The resulting slurry underwent immediate and continuous particle size reduction by processing with homogenizers. The homogenized slurry (i.e. comminuted seed mixture 311) was immediately fed into a centrifuge decanter with sufficient force to enable separation of solid phase 316 and liquid phase 317.

Solid phase 316 had a moisture content of 50.8% and a dry-basis composition: 4.5% (w/w) oil, 36.6% (w/w) protein, 6.4% (w/w) ash and 52.6% (w/w) carbohydrate. The corresponding decanter liquid phase 317 comprising a mixture of liquefied seed components, served to direct the majority of the seed oil (>85%) away from the protein production pathway. The solid phase 320 was combined in a holding tank while maintaining constant mixing of the contents. The pH of the solid phase slurry was adjusted. Following extraction, the solid phase extract 321 was fed into a centrifuge decanter with sufficient force on the solid phase extract 321 to separate a second solid phase 326 and a second liquid phase 327. Separation of the slurry in the decanter occurred in 28 minutes. The second solid phase 326 (69.8 kg) had a moisture content of 60.4% and a dry-basis composition: 2.6% (w/w) oil, 15.0% (w/w) protein, 8.1% (w/w) ash and 74.4% (w/w) carbohydrate. The second liquid phase 327 (337.1 kg) had a moisture content of 96.5% and a dry-basis composition: 9.7% (w/w) oil, 90.6% (w/w) protein, 6.9% (w/w) ash and no detected carbohydrate.

The pH of the second liquid phase 327 was reduced to 5.0 and the resulting mixture was separated in a clarifier centrifuge. A sample of the resulting pellet or protein precipitate 346 was dried to produce a first protein isolate 351 from example process 300 and subprocess 300b with the composition shown in Table 14.

TABLE 14
First protein isolate: 351

| Composition: | |
|---|---|
| Crude Protein (% as-is) (N × 6.25) | 93.06 |
| Moisture (%) | 2.6 |
| Crude Fat (%) | 6.56 |
| Crude Ash (%) | 4.32 |
| Carbohydrate (by difference, %) | — |

The total oil and protein in protein isolate 351 comprised a total 99.62% of the material, in a protein to oil ratio of 14.2:1.

### Example 8—Protein Solubility

This example illustrates the protein solubility of the hemp protein products prepared according to the present invention as described in Examples 2, 4 and 5. Protein solubility was tested by a modified version of the procedure of Morr et al., 1985, J. Food Sci., 50: 1715-1718. 2.0 g of protein powder were dispersed in 200 mL of reverse osmosis (RO) water after which, the mixture was stirred until a smooth paste formed. Then, the solution pH was adjusted to a selected level 2, 3, 4, 5, 6, or 7 using 10N HCl, 85% H3PO4 or 50% NaOH. Solutions were then stirred at 500 rpm using an overhead mixer for 60 minutes at 4° C. to facilitate protein solubility. Samples were left static for 10 min to allow aggregates to precipitate, then transferred to 50 mL falcon tubes and centrifuged for 15 min at room temperature (25° C.) at 3,000×g. After centrifugation, the protein content of the supernatant was measured by combustion using a Flashsmart protein analyzer (% Nitrogen×6.25). The percent protein solubility value was determined based on division of the protein content value in the supernatant by the protein content value in the initial sample (×100).

Protein Solubility (%)=(% protein in supernatant/% protein in initial dispersion)×100

The protein solubility of the products at different pH values is shown in Table 13.

TABLE 15
Protein solubility

| | Solubility (%) | | | | | |
|---|---|---|---|---|---|---|
| Product | pH2 | pH3 | pH4 | pH5 | pH6 | pH7 |
| 231 (Example 2) | 62.0 | 65.4 | 59.8 | 46.2 | 13.5 | 1.1 |
| 341 (Example 5) | 57.4 | 52.9 | 50.6 | 39.4 | 2.3 | 34.9 |
| 341 (Example 6) | 60.8 | 64.2 | 49.3 | 45.9 | 1.2 | 29.8 |

### Example 9—Oil-Holding Capacity and Water-Hydration Capacity

This Example illustrates the oil-holding capacity (OHC) and water-hydration capacity (WHC) of the hemp protein products prepared according to the present invention as described in Examples 2, 4 and 6. OHC and WHC were tested by a modified version of the procedure of Stone et al., 2015, Food Sci. Biotechnol., 27: 827-833. 0.25 g of protein product was transferred into a pre-weighed 50 mL centrifuge tube. Then, 10 mL of hemp oil or reverse-osmosis water were added, followed by vortexing (S/P® Vortex Mixer; VWR Inc.) for 10 sec every 5 min for a total of 30 min. Samples underwent centrifugation at 1000 rpm using an Eppendorf centrifuge 5810 for 15 min. The supernatant was decanted after which, the tube and sediment were weighed. For WHC, the tube was placed upside down for 10 min prior to weighing. OHC and WHC values were calculated in g (oil or water)/g of protein product using the following equation:

OHC or WHC=(Wet sample weight−Dry sample weight)/Dry sample weight

TABLE 16
Oil-holding capacity and Water-hydration capacity

| Product | OHC | WHC |
|---|---|---|
| 231 (Example 2) | 1.00 | 1.57 |
| 341 (Example 5) | 1.20 | 1.80 |
| 341 (Example 6) | 0.80 | 1.52 |

### Example 10—Emulsion Stability

This example illustrates the emulsion stability (ES) of the hemp protein products prepared according to the present invention as described in Examples 2, 4, and 6. ES was tested by a modified version of the procedure of Galves et al., 2019, Cereal Chem., 96: 1036-1047. The ES of a dispersion of the protein products (0.25% w/w) was determined by homogenizing 40 mL of protein solution with 20 mL of hemp oil using a L5M-A Laboratory Mixer Silverson brand homogenizer positioned near the oil-water interface at 10,260 rpm for 10 min. The emulsion formed was immediately transferred to a 50-mL graduated cylinder and observed for separation of the aqueous phase from the turbid phase of the emulsion after 30 min. The emulsion stability was calculated by equation below:

$$E S = \frac{{V}_{B} - {V}_{A}}{{V}_{B}} \times 100 \%$$

where V_{B} is the volume of the aqueous phase before homogenization (40 mL) and VA is the volume of the aqueous phase 30 minutes after homogenization.

TABLE 17
Emulsion stability

| Product | ES (%) |
|---|---|
| 231 (Example 2) | 51.3 |
| 341 (Example 5) | 56.3 |
| 341 (Example 6) | 53.0 |

### Example 11—Foaming Properties

This example illustrates the foaming capacity (FC) and stability (FS) of the hemp protein products prepared according to the present invention as described in Examples 2, 4 and 6. The FC and FS were tested by a modified version of the procedure of Galves et al., 2019. Foam was produced by vortexing (S/P® Vortex Mixer; VWR Inc.) 1% (w/w) dispersion of hemp protein products. In brief, 0.2 g of protein product was dispersed in 20 g of a 10 mM sodium phosphate buffer, then the solution pH was adjusted to 3.0 with 85% H3PO4 followed by vortexing for 3 min. After vortexing, the Foam Volume (FV0) was measured to give foam capacity (FC), calculated by the following equation:

$$F C = \frac{{V}_{F 0}}{{V}_{sample}} \times 100 \%$$

The Foaming Stability (FS) was calculated by followed equation:

$$F S = \frac{{V}_{F 30}}{{V}_{F 0}} \times 100 \%$$

where FV₀ is the foam volume at to after foaming and FV₃₀ is the foam volume at t=30 min. The foaming capacity of a protein is measured as the amount of interfacial area that can be created by vortexing the protein solution. Foam stability is measured as the time required to lose either x % of the volume from the foam.

TABLE 18
Foaming Capacity and Stability

| Product | FC (%) | FS (%) |
|---|---|---|
| 231 (Example 2) | 15 | 91.7 |
| 341 (Example 5) | 15 | 100 |
| 341 (Example 6) | 15 | 91.7 |

### Example 12—Colour

This example illustrates the colorimetric characterization of the hemp protein products prepared according to the present invention as described in Examples 2, 4 and 6. L* is normalized to the values 0 to 100 corresponding to a percentage scale which describes the lightness of a sample. L*=100% means 100% light and L*=0 means no light (black). Positive a* values represent reddish tones and negative values greenish tones. A more positive a* value indicates that the tone is more reddish. A more negative a* value indicates that the tone is more greenish. Positive b* values represent yellowish tones and negative b* values represent blueish tones. A more positive b* value indicates that the tone is more yellowish. A more negative b* value indicates that the tone is more blueish. The color measurements of each sample were captured by a Spectro 1™ colorimeter device (available from Variable Inc., 10 Chattanooga, Tenn., USA) by pointing the device at a container containing a sample and then recording the color of the target sample.

TABLE 19
Colour

| Product | L* | a* | b* |
|---|---|---|---|
| 231 (Example 2) | 56.8 | 2.3 | 14.6 |
| 341 (Example 5) | 60.2 | 0.8 | 13.4 |
| 341 (Example 6) | 63.0 | 0.7 | 9.2 |

### Example 13—Preparation of Another Hemp Protein-Lipid Complex Containing Mixture

This example refers to process 200 depicted in FIG. 2A and FIG. 2B. Liquid phase 217 comprising a mixture of seed components, was made according to the process described in Example 3. A portion of the liquid phase was spray dried to provide second protein-lipid complex containing mixture 236 from process 200.

TABLE 20
Second protein-lipid complex containing mixture 236

| Composition: | |
|---|---|
| Crude Protein (%) (N × 6.25) | 33.6 |
| Moisture (%) | 3.2 |
| Crude Fat (%) | 59.3 |
| Crude Ash (%) | 2.7 |
| Carbohydrate (%, by difference) | 1.2 |

The total oil and protein in protein-lipid complex containing mixture 236 comprised a total 92.9% of the material in a protein to oil ratio of about 0.6:1.

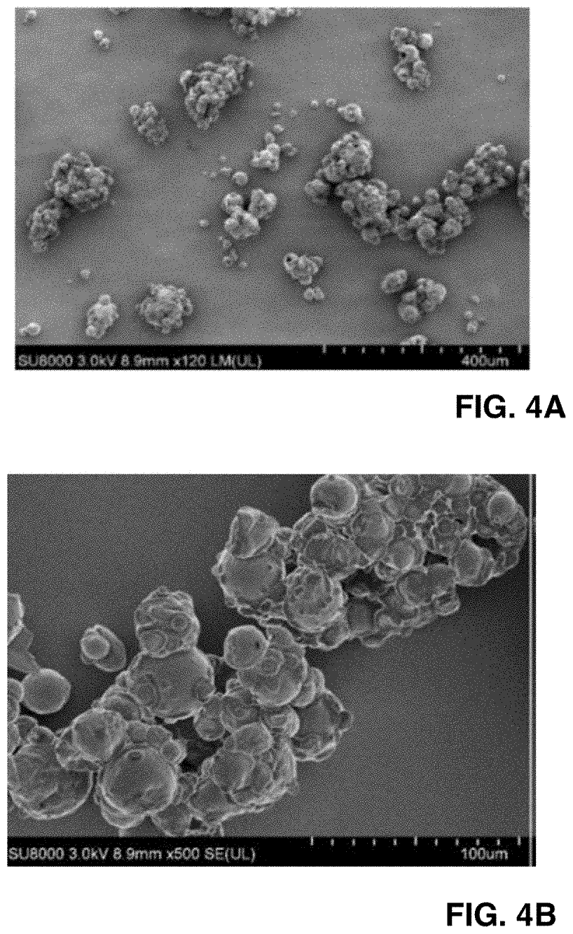

The protein-lipid composition protein-lipid complex containing mixture 236 can be described as a free-flowing powder with a low bulk density (0.26 kg/m³). Scanning electron microscopy images of the spray dried material corroborated the low bulk density measurement as the material was observed to exhibit porosity (see: FIGS. 4A,18 4B). The inventors consider the porous microstructure of protein-lipid complex containing mixture 236 surprising since in protein-lipid complex containing mixtures comprising high concentration of lipids, such as 59.3% in the current example mixture, it is expected that protein containing aggregates are formed, and not porous microstructures, as was observed.

The microstructure exhibited by protein-lipid complex-containing mixture 236 may make the material suitable for use in the preparation of a variety formulations, for example, formulations in which a high-water-hydration capacity is desirable, in formulations in which a high oil-holding capacity is desirable, in formulations in which foaming capacity is desirable, or in formulations in which an emulsifying agent is desirable. As shown in Table 21, protein-lipid complex containing mixture 236 had high oil-holding water-hydration and foam-forming capacities, and may act as an emulsifier.

TABLE 21
Functional properties of protein-lipid
complex containing mixture 236

| | Water- | | Emulsion | Emulsion |
|---|---|---|---|---|
| Oil-Holding | Hydration | Foaming | Stability | Stability |
| Capacity: | Capacity | Capacity | T = 30 min | T = 24 h |
| 1.25 | 0.59 | 198.3% | 100% | 59.0% |

1. A hemp protein composition comprising from about 30% (w/w) and to about 95% (w/w) hemp protein (N×6.25), from about 5% (w/w) and to about 60% (w/w) plant oil.

2. The hemp protein composition according to claim 1 which additionally comprises up to about 4% (w/w) carbohydrates.

3. The hemp composition according to claim 1 which additionally comprises from about 1% (w/w) and to about 4% (w/w) carbohydrates.

4. The hemp protein composition according to claim 1, comprising from about 34% (w/w) and to about 93% (w/w) hemp protein (N×6.25), from about 7% (w/w) and to about 59% (w/w) plant oil, and from about 1% (w/w) and to about 4% (w/w) carbohydrates.

5. The hemp protein composition according to claim 1, wherein hemp protein compositions, is a hemp protein concentrate comprising from at least about 84% (w/w) and less than about 90% (w/w) hemp protein (N×6.25), and from about 11% (w/w) to about 16% (w/w) plant oil.

6. The hemp protein composition according to claim 5, wherein the hemp protein concentrate comprises at least about 85% (w/w) and less than about 90% (w/w) hemp proteins (N×6.25).

7. (canceled)

8. The hemp protein composition according to claim 1, wherein the hemp protein composition is a hemp protein isolate comprising at least about 90% (w/w) hemp protein (N×6.25), and from about 6% (w/w) to about 8% (w/w) plant oil.

9. (canceled)

10. (canceled)

11. The hemp protein composition according to claim 8, wherein the hemp protein isolate comprises at least about 93% (w/w) hemp proteins (N×6.25).

12. The hemp protein composition according to claim 1, wherein the hemp protein composition is a hemp protein-lipid complex containing mixture comprising from about 34% (w/w) to about 49% (w/w) protein (N×6.25), from about 34% (w/w) to about 59% (w/w) plant oil, and from about 1% (w/w) to about 4% (w/w) carbohydrates.

13. The hemp protein composition according to claim 1, wherein the total protein (N×6.25) and plant oil content may range from about 83% to about 100%, and wherein the ratio of protein to oil may range from about 1:1 to about 10:1 on a weight-by-weight basis.

14. The hemp protein composition according to claim 1, wherein the hemp protein composition is a hemp protein concentrate comprising a total protein (N×6.25) and plant oil content of about 100% (w/w).

15. The hemp protein composition according to claim 1, wherein the hemp protein compositions is a hemp protein isolate comprising a total protein (N×6.25) and plant oil content of about 100% (w/w).

16. The hemp protein composition according to claim 1, wherein the hemp protein composition is a hemp protein-lipid complex containing mixture comprising a total protein (N×6.25) and plant oil content of at least about 83% (w/w).

17. The hemp protein composition according to claim 1, wherein the hemp protein composition is a hemp protein concentrate, wherein the ratio of protein (N×6.25) to oil is at least about 5.3:1 on a weight-by-weight basis.

18. The hemp protein composition according to claim 1, wherein the hemp protein composition is a hemp protein isolate, wherein the ratio of protein (N×6.25) to oil is at least about 14:1 on a weight-by-weight basis.

19. The hemp protein composition according to claim 1, wherein the hemp protein composition is a hemp protein-lipid complex containing mixture, wherein the ratio of protein (N×6.25) to oil is at least about 0.6:1 on a weight-by-weight basis.

20. The hemp protein composition according to claim 1, wherein the weight percentage of essential amino acids in the hemp protein composition is about 30% (w/w) or more by weight protein.

21. The hemp protein composition according to claim 1, wherein the lysine content ranges from about 2.5% to about 5% by weight of crude protein of the hemp protein concentrates.

22. A method of making a hemp protein composition comprising the steps of:
(i) providing seeds from a hemp plant;
(ii) comminuting the seeds in an aqueous solution to obtain a mixture comprising comminuted seed particles having mean particle sizes in a range of about 5 μm to about 200 μm; and
(iii) separating the mixture into a first solid phase and a first liquid phase; and at least one of:
(A)
(iv) separating the first liquid phase into a light liquid phase and a heavy liquid phase; and
(a)
(v) treating the heavy liquid phase by microfiltration to recover therefrom a permeate;
(vi) treating the permeate by ultrafiltration to recover therefrom the protein retentate; and
(vii) drying the protein retentate to obtain a first protein-lipid complex containing mixture;
or
(b)
(viii) precipitating the protein in the heavy liquid phase to obtain a protein precipitate; and
(ix) drying the protein precipitate to obtain a first protein concentrate;
or
(B)
(x) extracting the first solid phase to obtain a first solid phase extract;
(xi) separating the first solid phase extract to obtain a second solid phase and a second liquid phase; and
(a)
(xii) settling the second liquid phase to obtain a heavy second liquid phase and a light oil-rich phase;
(xiii) precipitating the heavy second liquid phase to recover a protein precipitate; and
(xiv) drying the protein precipitate to obtain a third protein concentrate;
or
(b)
(xv) precipitating the protein in the second liquid phase to recover a protein precipitate; and
(xvi) drying the protein precipitate to obtain a protein isolate;
or
(C)
(xvii) drying the first liquid phase to obtain a second protein-lipid complex containing mixture.

23. (canceled)

24. (canceled)

25. (canceled)

26. (canceled)

27. (canceled)

28. (canceled)

29. (canceled)

30. (canceled)

31. A nutritional formulation comprising a hemp protein composition comprising at least about 65% (w/w) or more plant protein, from about 30% (w/w) to about 95% hemp protein (N×6.25), from about 5% (w/w) and to about 60% (w/w) plant oil, the balance of the hemp protein composition substantially being constituted by carbohydrates, water and ash.
